(19) United States
(12) Patent Application Publication
(10) Pub. No.: US 2025/0213677 A1
(43) Pub. Date: Jul. 3, 2025

(54) DRUG-DELIVERY COMPOSITION INCLUDING PLANT-DERIVED NANOVESICLES AS ACTIVE INGREDIENTS

(71) Applicant: Andong National University Industry-Academic Cooperation Foundation, Andong-si (KR)

(72) Inventor: Young Eun CHO, Andong-si (KR)

(73) Assignee: Andong National University Industry-Academic Cooperation Foundation, Andong-si (KR)

(21) Appl. No.: 18/611,577

(22) Filed: Mar. 20, 2024

(30) Foreign Application Priority Data
Dec. 28, 2023 (KR) 10-2023-0194044

(51) Int. Cl. A61K 39/29; A61K 9/51; A61K 39/00; A61K 39/145; A61P 31/20; B82Y 5/00; C12N 7/00

(52) U.S. Cl. CPC A61K 39/29; A61K 9/5176; A61K 39/145; A61P 31/20; C12N 7/00; A61K 2039/55555; B82Y 5/00; C12N 2710/00034; C12N 2760/16134

(57) ABSTRACT

Proposed is a drug-delivery composition including plant-derived nanovesicles as an active ingredient. It was confirmed that antigen (drug) delivery efficiency is higher when the antigens are loaded into nanovesicles isolated from grapefruits or mandarin oranges, and the loaded antigens are administered rather than administering hepatitis B virus surface antigens (HBsAg) alone. Thus, the nanovesicles can be useful as a drug-delivery composition.

### STATEMENT REGARDING GOVERNMENT SPONSORED RESEARCH

This invention is a result of a study on the “Leaders in Industry-university Cooperation 3.0 (LINC 3.0)” Project, supported by the 15 Ministry of Education and the National Research Foundation of Korea (NRF) [Project number; LINC3.0-2023-0425; Project serial number: 1345370672].

### CROSS REFERENCE TO RELATED APPLICATION

The present application claims priority to Korean Patent Application No. 10-2023-0194044, filed Dec. 23, 2023, the entire contents of which is incorporated herein for all purposes by this reference.

### BACKGROUND OF THE DISCLOSURE

### 1. Field of the Disclosure

The present disclosure relates to a drug-delivery composition including plant-derived nanovesicles as an active ingredient.

### 2. Description of the Related Art

Vaccination has been considered the most cost-effective method to control a variety of infectious diseases caused by viruses and pathogenic microorganisms. As seen with the current COVID-19 pandemic, the absence of effective vaccines against newly emerging viruses could seriously threaten public health worldwide. Recombinant protein vaccines, mRNA vaccines, and viral vector vaccines have been designed as next-generation vaccine platforms in addition to classic types of vaccines such as inactivated vaccines and live attenuated vaccines. Besides vaccine platforms, developing new vaccine delivery tools is also important to improve vaccine efficacy through improved antigen delivery to immune cells.

Meanwhile, extracellular vesicles (hereinafter referred to as EVs) are small vesicles that produce various types of cells. EVs have emerged as a new delivery system for small interfering RNA (siRNA), pharmaceutically active substances, and even vaccine antigens. Plants release nanovesicles similar to exosomes. It has been reported that the nanovesicles play a role in communication between plant cells and exert various biological activities by delivering miRNA, mRNA, and proteins. In addition, while mammalian-derived nanovesicles are difficult to mass-produce, plant-derived exosome-like nanovesicles (PENVs) can be easily isolated and purified in large quantities and are non-toxic and stable because PENVsare obtained from edible plants. Despite the advantages of plant-derived exosome-like nanovesicles, research on the use of PENVs as a new delivery tool is still insufficient.

### DOCUMENT OF RELATED ART

### Patent Document

(Patent Document 1) 1. Korean Patent Registration No. 10-2065819, filed on Jan. 7, 2020

### SUMMARY OF THE DISCLOSURE

The present disclosure is to provide a drug-delivery composition including plant-derived nanovesicles as an active ingredient.

For this, the present disclosure provides a drug-delivery composition including nanovesicles derived from grapefruits (Citrus×paradisi) or mandarin oranges (Citrus reticulata) as an active ingredient.

In addition, the present disclosure provides a pharmaceutical composition for preventing or treating hepatitis B, the pharmaceutical composition including nanovesicles, the nanovesicles being loaded with a hepatitis B treatment drug as an active ingredient.

In addition, the present disclosure provides a health functional food composition for preventing or improving hepatitis B, the health functional food composition including nanovesicles, the nanovesicles being loaded with a hepatitis B treatment drug as an active ingredient.

According to the present disclosure, it was confirmed that antigen (drug) delivery efficiency is higher when the antigens are loaded into nanovesicles isolated from grapefruits or mandarin oranges, and the loaded antigens are administered rather than administering hepatitis B virus surface antigens (HBsAg) alone. Thus, nanovesicles can be useful as a drug-delivery composition.

### BRIEF DESCRIPTION OF THE DRAWINGS

FIGS. 1A and 1B show the results of analyzing the size and shape of nanovesicles derived from grapefruits (hereinafter referred to as GNVs) and nanovesicles derived from mandarin oranges (hereinafter referred to as MNVs), and the GNVs and MNVs are hereinafter referred to as samples;

FIGS. 2A and 2B show the results of analyzing the cytotoxicity of samples in kidney cells, and data are expressed as mean±error (SEM) for three independent experiments;

FIGS. 3A and 3B show the results of analyzing whether the samples labeled with 1,1′-dioctadecyl-3,3,3′,3′-tetramethylindotricarbocyanine iodide (DiRDiR, DiIC18(7)) are well absorbed in kidney cells/organs;

FIGS. 4A to 4F show the results of analyzing the drug-delivery ability of the samples; and

FIGS. 5A and 5B show the results of analyzing the effect of the samples on immunogenicity in drug-delivery.

### DESCRIPTION OF THE PREFERRED EMBODIMENTS

Hereinafter, the present disclosure will be described in more detail.

The present disclosure provides a drug-delivery composition including nanovesicles derived from grapefruits (Citrus×paradisi) or mandarin oranges (Citrus reticulata) as an active ingredient.

The nanovesicles derived from grapefruits may have a size in a range of 30 nm to 60 nm. The nanovesicles derived from mandarin oranges may have a size in a range of 100 nm to 300 nm.

The nanovesicles can improve drug-delivery efficiency into cells, and the drug may be hepatitis B virus surface antigens (HBsAg) or influenza virus antigens but are not limited thereto.

In addition, the present disclosure provides a pharmaceutical composition for preventing or treating hepatitis B, the pharmaceutical composition including nanovesicles, the nanovesicles being loaded with a hepatitis B treatment drug as an active ingredient.

The hepatitis B treatment drug may be hepatitis B virus surface antigens (HBsAg).

According to a method that can be easily carried out by those ordinarily skilled in the art to which the disclosure pertains, the pharmaceutical composition of the present disclosure can be formulated by using pharmaceutically acceptable carrier, and then can be prepared in unit dosage form or can be prepared by placing the pharmaceutical composition in a multi-dose container.

The pharmaceutically acceptable carriers are ones commonly used in preparation. The pharmaceutically acceptable carriers include but are not limited to lactose, dextrose, sucrose, sorbitol, mannitol, starch, gum acacia, calcium phosphate, alginate, gelatin, calcium silicate, microcrystalline cellulose, polyvinylpyrrolidone, cellulose, water, syrup, methyl cellulose, methyl hydride. roxybenzoate, propylhydroxybenzoate, talc, magnesium Stearate, and mineral oil. In addition to the components, the pharmaceutical composition of the present disclosure may further include lubricants, wetting agents, sweeteners, flavoring agents, emulsifiers, suspending agents, and preservatives.

In the present disclosure, the content of additives included in the pharmaceutical composition is not particularly limited and can be appropriately adjusted within the content range used in conventional formulations.

The pharmaceutical composition may be formulated in the form of one or more external skin preparations selected from the group consisting of injectable formulations (for example, aqueous solutions, suspensions, emulsions), pills, capsules, granules, tablets, creams, gels, patches, sprays, ointments, warning agents, lotions, liniment agents, pasta agents, and cataplasma agents, but is not limited thereto.

The pharmaceutical composition of the present disclosure may further include pharmaceutically acceptable carriers and diluents for formulation. The pharmaceutically acceptable carriers and diluents include excipients (for example, starches, sugars, and mannitol), fillers and extenders (for example, calcium phosphate), cellulose derivatives (for example, carboxymethylcellulose and hydroxypropylcellulose), binders (for example, gelatin, alginate, and polyvinyl pyrrolidone), lubricants (for example, calcium stearate, hydrogenated castor oil, and polyethyleneglycol), disintegrants (for example, povidone and crospovidone), and surfactants (for example, polysorbate, cetylalcohol, and glycerol), but are not limited thereto. The pharmaceutically acceptable carriers and diluents may be biologically and physiologically friendly to a subject. The diluents may include saline, aqueous buffers, solvents, and/or dispersion media, but are not limited thereto.

The pharmaceutical composition of the present disclosure can be administered orally or parenterally (for example, intravenously, subcutaneously, intraperitoneally, or topically) depending on the desired method. For oral administration, the pharmaceutical composition may be formulated into tablets, troches, lozenges, aqueous suspensions, oily suspensions, powders, granules, emulsions, hard capsules, soft capsules, syrups, or elixirs. For parenteral administration, the pharmaceutical composition may be formulated into an injection, suppository, powder for respiratory inhalation, aerosol for spray, ointment, powder for application, oil, or cream.

The dosage range of the pharmaceutical composition of the present disclosure may vary depending on a patient's condition, weight, age, gender, health status, dietary constitution specificity, nature of preparation, degree of diseases, administration time of composition, administration method, administration period or interval, excretion rate, and drug form. The dosage can be appropriately selected by those skilled in the art. For example, the dosage may range from about 0.1 mg/kg to 10,000 mg/kg but is not limited thereto, and the pharmaceutical composition may be administered once or in divided doses several times a day.

The pharmaceutical composition can be administered orally or parenterally (for example, intravenously, subcutaneously, intraperitoneally, or topically) depending on the desired method. The pharmaceutically effective amount and effective dosage of the pharmaceutical composition of the present disclosure may vary depending on formulation methods, administration methods, administration time, and administration routes of the pharmaceutical composition. Those skilled in the art can easily determine and prescribe an effective dosage for the desired treatment. The pharmaceutical composition of the present disclosure may be administered once a day or may be administered in several divided doses.

In addition, the present disclosure provides a health functional food composition for preventing or improving hepatitis B, the health functional food composition including nanovesicles, the nanovesicles being loaded with a hepatitis B treatment drug as an active ingredient.

The hepatitis B treatment drug may be hepatitis B virus surface antigens (HBsAg).

The present disclosure can be generally used as commonly used foods.

The health functional food composition of the present disclosure can be used as health functional foods. The “health functional foods” refers to foods manufactured and processed using raw materials or ingredients with functional properties useful to the human body in accordance with the Health Functional Foods Act. “Functional” implies an intake of foods for the purpose of controlling nutrients necessary for the structure and function of the human body or for the purpose of obtaining useful health effects such as physiological effects.

The health functional food composition may include common food additives. Unless otherwise specified, suitability as a “food additive” is determined in accordance with the specifications and standards of the item under the general provisions and general test methods of the Food Additives Code approved by the Ministry of Food and Drug Safety.

The items listed in the “Food Additives Code” may include chemical compounds (for example, ketones, glycine, potassium citrate, nicotinic acid, and cinnamic acid), natural additives (for example, dark pigment, licorice extract, crystalline cellulose, high-quality pigment, and guar gum), and mixed preparations (for example, L-sodium glutamate preparation, noodle additive alkali preparation, preservative preparation, and tar coloring preparation).

The health functional food composition of the present disclosure can be manufactured and processed in the form of tablets, capsules, powders, granules, liquids, and pills. For instance, among health functional foods in capsule form, hard capsules can be produced by mixing the composition of the present disclosure with additives such as excipients and filling the hard capsule with the mixed composition. Meanwhile, soft capsules can be produced by mixing the composition of the present disclosure with additives such as excipients and filling a capsule base such as gelatin with the mixed composition. The soft capsules may contain plasticizers such as glycerin or sorbitol, colorants, and preservatives, if necessary.

The definitions of terms such as excipients, binders, disintegrants, lubricants, coagulants, and flavoring agents are described in literature known in the art and include those with the same or similar functions. There is no particular limit to the type of foods, and the foods include all health functional foods in the conventional sense.

In the present disclosure, the term “prevention” refers to all actions that suppress or delay diseases by administering the composition according to the present disclosure.

In the present disclosure, the term “treatment” refers to any action that improves or beneficially changes the symptoms of diseases by administering the composition according to the present disclosure.

In the present disclosure, the term “improvement” refers to all actions that improve the bad condition of diseases by administering the composition according to the present disclosure.

Hereinafter, the present disclosure will be described in detail through examples to aid understanding. However, the following examples only illustrate the content of the present disclosure, and the scope of the present disclosure is not limited to the following examples. Examples of the present disclosure are provided to more completely explain the present disclosure to those skilled in the art.

### [Experiment Example 1] Experiment Preparation

### 1-1. Samples

Grapefruits (Citrus×paradisi) and Mandarin oranges (Citrus reticulata) were obtained from Israel and Korea (Jeju Island). The grapefruits and mandarin oranges were washed under distilled water, peeled, and mixed with cold PBS (phosphate-buffered saline) to obtain fruit juice. The fruit juice was sequentially centrifuged, and a pellet obtained from the centrifugation was resuspended in PBS to prepare nanovesicles derived from grapefruits (GNVs) and nanovesicles derived from mandarin oranges (MNVs) to be used as the samples of the present disclosure. Aliquots of the samples were stored at a temperature of −80° C. until use. The protein concentration of the GNVs and MNVs was measured by BCA analysis.

### 1-2. Cell Models

Madin-Darby Canine Kidney (MDCK) cells and Vero cells (cells derived from kidney epithelial cells of African green monkeys) for use were purchased from ATCC. The cells were seeded in Dulbecco's Modified Eagle Medium (DMEM, Hyclone Laboratories, Inc., Logan, UT, USA) containing 10% fetal bovine serum (FBS) and 1% penicillin, respectively, and the cells were cultivated under the following conditions: a 5% CO₂ atmosphere and a temperature of 37° C.

### 1-3. Animal Models

Six-week-old male BALB/c mice for use were purchased from Koatech (Pyeongtaek, Korea). The mice were allowed to freely consume food and water under a 12-hour light/dark cycle.

### [Experiment Example 2] Nanoparticle Tracking and Transmission Electron Microscopy Analysis

The size and shape of samples were analyzed through nanoparticle tracking analysis (NTA) with Nanosight NS300 (Malvern Instruments, Malvern, UK) and negative staining transmission electron microscopy (hereinafter referred to as TEM). The images of the samples were visualized using TEM. The images of the samples were obtained by staining the samples with 0.75% uranyl formate and coating the samples on a glow-discharged copper grid. The samples were examined using a JEM-1010 transmission electron microscope (JEOL, Tokyo, Japan) at an acceleration voltage of 100 kV.

### [Experiment Example 3] MTT Assay

To confirm the cytotoxicity of samples in kidney cells, an MTT assay was performed. MDCK and Vero cells were dispensed into 96-well plates and cultivated under the conditions of Experiment Example 1-2. After 24 hours, cells were treated with the samples at different concentrations and cultivated for 24 hours. Afterward, cytotoxicity was analyzed using 3-[4,5-dimethylthiazol-2-y]-2,5-diphenyltetrazolium bromide (MTT, Sigma, St. Louis, MO, USA). Formazan was dissolved in dimethyl sulfoxide (DMSO). Absorbance was measured at a wavelength of 570 nm using a microplate ELISA reader (Infinite m200pro; Tecan, Gradig, Austria).

### [Experiment Example 4] Sample-DiR Labeling and Organ Imaging

Sample labeling was performed according to the manufacturer's instructions (ThermoFisher, D12731) by using 1,1′-Dioctadecyl-3,3,3′,3′-tetramethylindotricarbocyanine iodide (DiR, DiIC18 (7)). Briefly, 50 μg/mL of samples was resuspended in 10 μL of 1 m MDiR stock solution and incubated for 30 minutes at room temperature. DIR-labeled samples were separated into pellets using an ultracentrifuge (100,000 g×1). The pellets were resuspended in 1 mL PBS, and the supernatant was discarded. Afterward, MDCK and Vero cells were cultivated with 1 μM DiR-labeled samples for 4, 8, and 12 hours. For nuclear staining, DAPI (Sigma Aldrich, USA) was mounted under a glass coverslip, and the slide was visualized through immunofluorescence microscopy.

For long-term imaging, the DiR-labeled samples were orally administered to the mice of Experiment Example 1-3 (1 mg protein, 1×10¹²/g body weight), and the organs of the mice were obtained after 4, 8, 12, and 24 hours of the administration. The obtained organs were excised and imaged with an Odyssey scanner (LI-COR, USA).

### [Experiment Example 5] Loading Proteins onto Samples

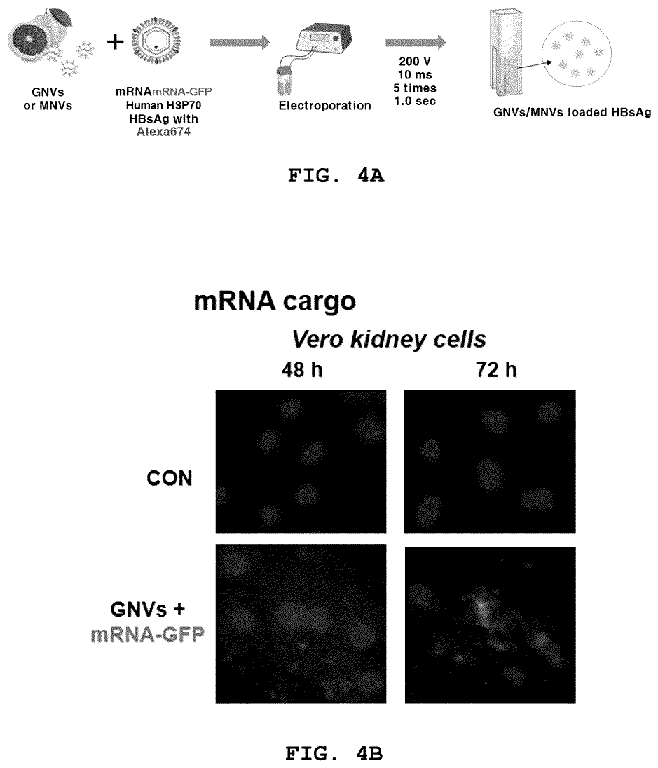

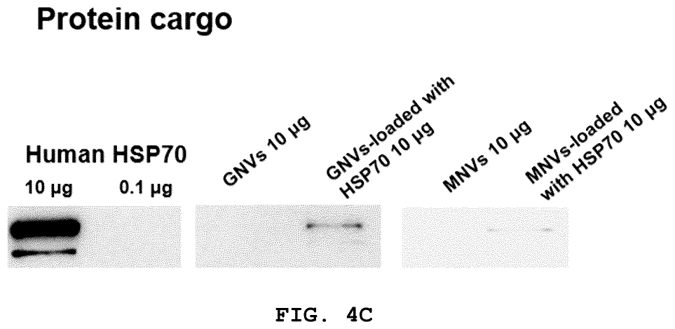

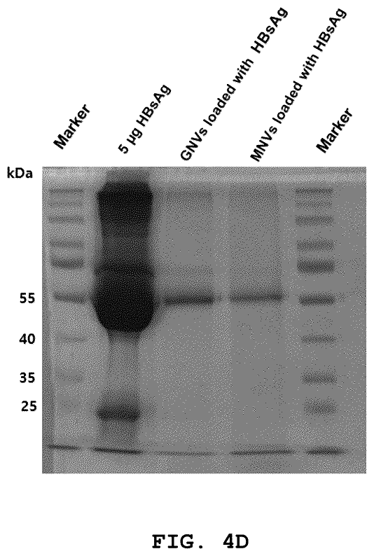

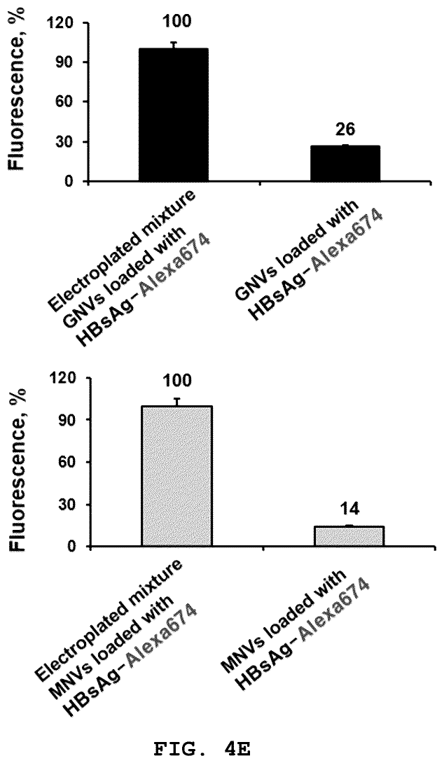

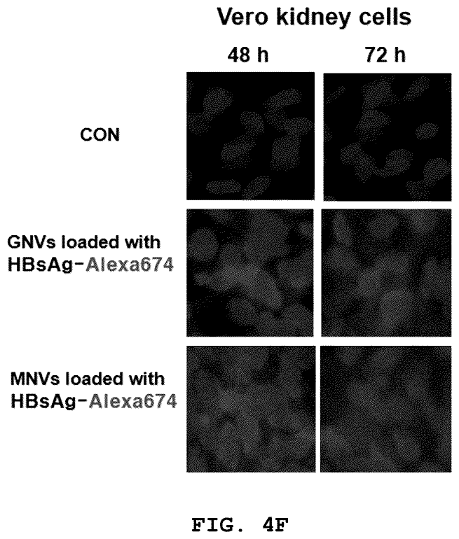

As shown in FIG. 4A, mRNA-green fluorescent proteins (mRNA-GFPs), heat shock protein 70 (human HSP70) proteins, and hepatitis B virus surface antigens (hereinafter referred to as HBsAg) recombinant protein were loaded onto samples through electrophoresis. Each sample loaded with the proteins was diluted in PBS (nanovesicle:PBS=1:9 weight ratio). mRNA-GFP (1 ng), human HSP70 protein (10 μg), and HBsAg recombinant protein (5 μg) were added to the buffer solution and then transferred to an ice-cold 0.4 cm Gene Pulser/MicroPulser cuvette (Electroporation Cuvette). Afterward, the electroporation cuvette was inserted into the Gene Pulser Xcell Total System, and electrophoresis was performed (voltage: 200 V, voltage difference: 10 ms, number of repetitions: 5, and voltage time: 1 second).

### [Experiment Example 6] Western Blotting Analysis

The same amount of proteins as the human HSP70 proteins loaded onto samples were separated by SDS-PAGE under reducing conditions. The proteins were transferred to a polyvinylidene fluoride (PVDF) membrane at a voltage of 120 V for 30 minutes. Blocking was performed for 2 hours at room temperature using 5% skim milk dissolved in PBS containing 0.1% Tween-20 (PBST), and the membrane was incubated overnight at a temperature of 4° C. with primary antibodies against HSP (abcam). Next, the membrane was incubated with horseradish peroxidase-conjugated secondary antibodies. Afterward, western blotting results were visualized using enhanced chemiluminescence (Super Pico Detection Reagent, Pierce: Rockford, IL, USA) and quantified using the ChemiDoc Gel Quantification System (Bio-Rad: Hercules, CA, USA).

### [Experiment Example 7] Vaccination to Animal Models

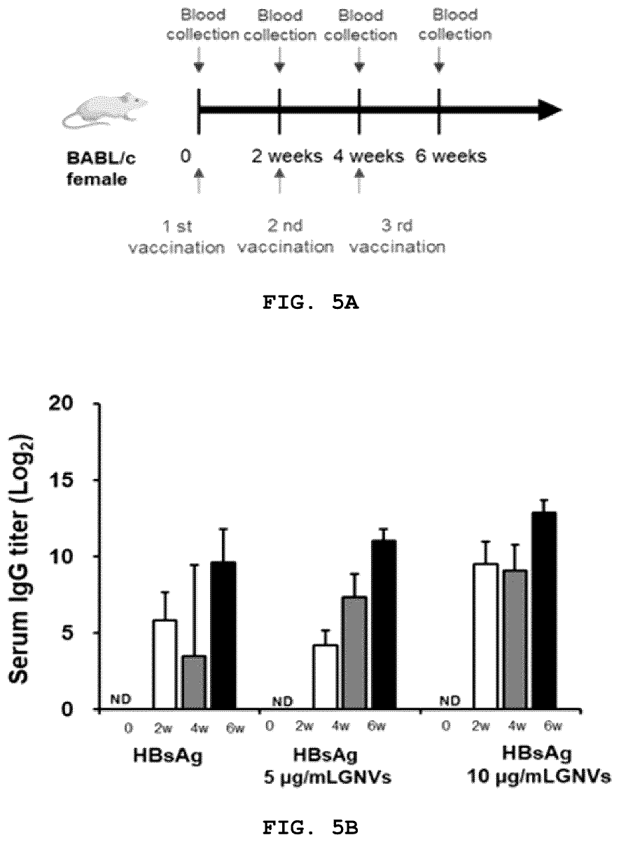

Six-week-old male BALB/c mice from Koatech (Pyeongtaek, Korea) were allowed to freely consume food and water under a 12-hour light/dark cycle. Next, as shown in FIG. 5A, HBsAg recombinant proteins loaded into GNVs were orally administered to the mice twice a week. In the case of the control (HBsAg), HBsAg recombinant proteins (10 μg/100 μL) not loaded into the nanovesicles (a conventional vaccine administration method) were intramuscularly administered into the mice a total of twice a week. Afterward, the mice were anesthetized, and the blood of the mice was collected through orbital blood collection.

### [Experiment Example 8] ELISA Assay

To determine the extent to which HBsAg was loaded into GNVs and MNVs in mouse serum, an ELISA analysis was performed. 96-well plates were coated with HBsAg proteins being loaded with samples for each well. After blocking, the plates were washed with 5-fold serially diluted serum for 1 hour at room temperature and incubated with horseradish peroxidase (HRP)-conjugated anti-mouse IgG antibodies for 1 hour at room temperature. After washing, the plates were incubated with a tetranethylbenzidine (TMB) solution for 5 minutes at room temperature. Reactions were stopped by adding 1N HCl solution, and absorbance was measured at a wavelength of 450 nm using an ELISA reader.

### [Experiment Example 9] Statistical Analysis

Data were analyzed using SPSS Statistics (ver. 27.0, SPSS Inc., Chicago, IL, USA). Statistical significance was set at the mean difference of p<0.05 using Tukey's HSD post-hoc analysis and one-way analysis of variance (ANOVA).

### [Example 1] Size and Shape Analysis of Samples

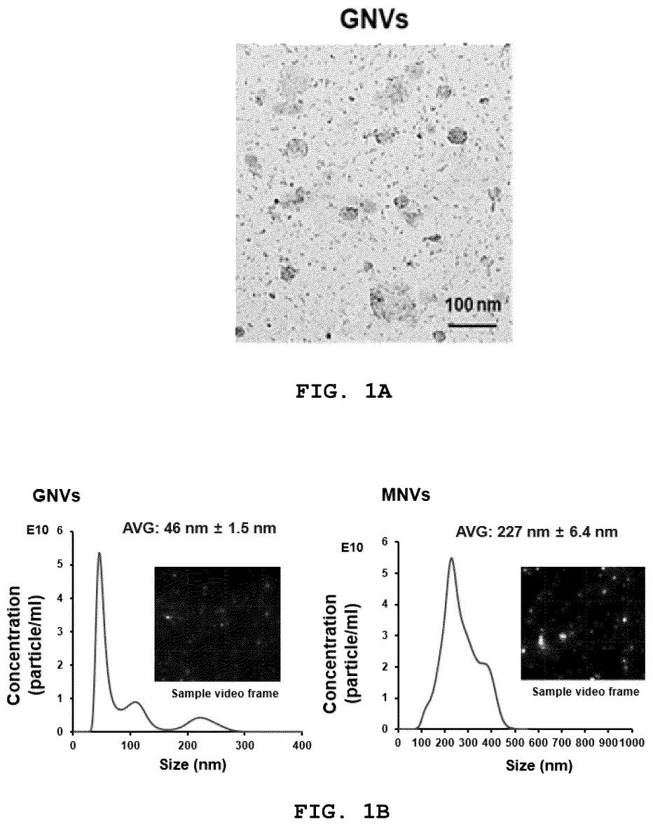

As a result of analyzing the size and shape of the samples according to Experiment Example 2, as shown in FIGS. 1A and 1B, the average size of GNVs and MNVs was 46±1.5 nm and 227±6.4 nm at 106 particles/mL, respectively, and the shape of the GNVs and MNVs were confirmed to be round with a size in a range of <100 nm (FIGS. 1A and 1B). From the results, it was confirmed that the GNVs and MNVs were successfully separated as nanovesicles.

### [Example 2] Cytotoxicity Analysis

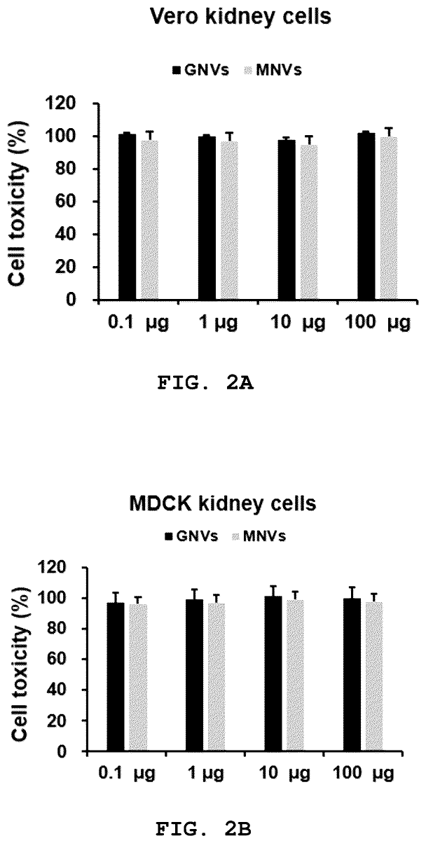

As a result of analyzing the cytotoxicity of the samples in kidney cells according to Experiment Example 3, as shown in FIGS. 2A and 2B, there was no significant change in cell viability in the sample treatment groups (FIGS. 2A and 2B). From the results, it was confirmed that GNVs and MNVs do not exhibit cytotoxicity in kidney cells.

### [Example 3] Analysis of Delivery Ability (Mobility) within Cells/Organs

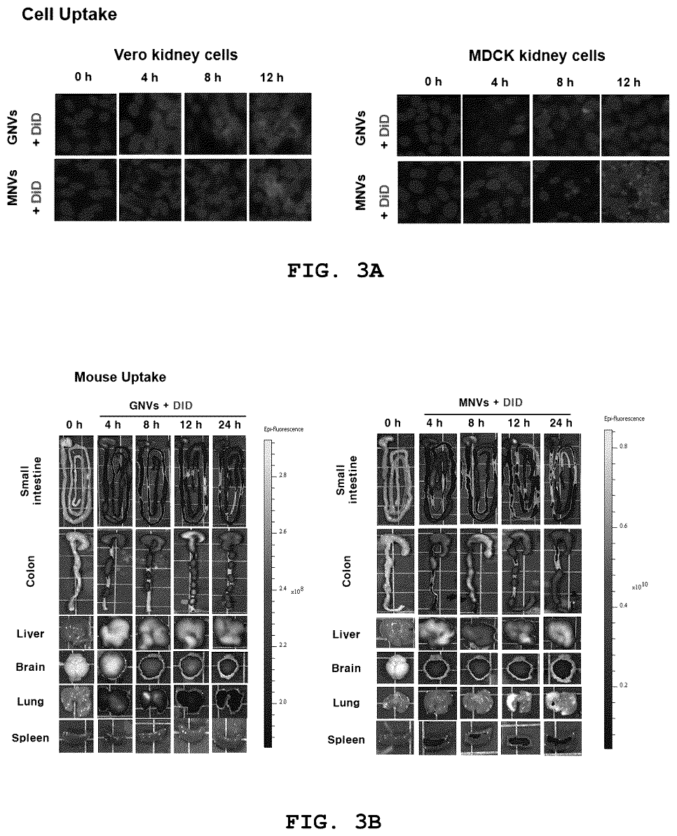

As a result of analyzing whether samples labeled with DiR were well absorbed in kidney cells/organs according to Experiment Example 4, as shown in FIGS. 3A and 3B, the samples were detected in kidney cells for 4 to 12 hours (FIG. 3A). Additionally, signals generated by the samples appeared in the small intestine, large intestine, liver, and brain 4 hours after oral administration of the samples to mice, and only in the liver 24 hours after the oral administration (FIG. 3B). From the results, it was confirmed that GNVs and MNVs labeled with DiR had excellent delivery ability (mobility) in cells/organs.

### [Example 4] Drug-Delivery Analysis

As a result of analyzing the drug delivery ability of the samples according to Experiment Examples 5 and 6, as shown in FIGS. 4A to 4F, in the electroporation analysis (Experiment Example 5), it was confirmed that the uptake of mRNA-GFP loaded into GNVs was more efficient compared to the control (CON; mRNA-GFP not loaded into GNVs) (FIG. 4B). In addition, western blotting analysis (Experiment Example 6) showed that the expression of HSP70 proteins loaded intothe GNVs was higher than that of HSP70 proteins loaded intothe MNVs (FIG. 4C). In addition, it was confirmed that the drug-delivery efficiency of HBsAg proteins loaded into GNVs was better than that of HBsAg proteins loaded into MNVs (FIGS. 4D to 4F). From the results, it was confirmed that the drug (HBsAg) delivery ability of the GNVs and MNVs was excellent, and among the two, the drug (HBsAg) delivery ability of the GNVs was more excellent.

### [Example 5] Immunogenicity Analysis of HBsAg

As a result of analyzing the effect of the sample-mediated delivery of HBsAg on immunogenicity in an animal model according to Experiment Examples 7 and 8, as shown in FIGS. 5A and 5B, it was confirmed that 5 or 10 μg of HBsAg loaded into the samples induced a higher level of antibodies compared to the control (10 μg of HBsAg not loaded into the samples) (FIG. 5B). From the results, it was confirmed that GNVs-mediated and MNVs-mediated delivery of HBsAg improved the antibody response to the antigens of HBsAg.

As above, specific parts of the present disclosure have been described in detail. For those skilled in the art, it is clear that these specific techniques are merely preferred examples and do not limit the scope of the present disclosure. That is, the practical scope of the present disclosure is defined by the appended claims and their equivalents.

What is claimed is:

1. A drug-delivery composition comprising nanovesicles derived from grapefruits (Citrus×paradisi) or mandarin oranges (Citrus reticulata) as an active ingredient.

2. The drug-delivery composition of claim 1, wherein the nanovesicles derived from grapefruits have a size in a range of 30 nm to 60 nm.

3. The drug-delivery composition of claim 1, wherein the nanovesicles derived from mandarin oranges have a size in a range of 100 nm to 300 nm.

4. The drug-delivery composition of claim 1, wherein the nanovesicles improve drugdelivery efficiency into cells.

5. The drug-delivery composition of claim 1, wherein the drug is a hepatitis B virus surface antigen (HBsAg) or influenza virus antigen.

6. A pharmaceutical composition for preventing or treating hepatitis B, the pharmaceutical composition comprising the nanovesicles of claim 1, the nanovesicles being loaded with a hepatitis B treatment drug as an active ingredient.

7. The pharmaceutical composition of claim 6, wherein the hepatitis B treatment drug is a hepatitis B virus surface antigen (HBsAg).

8. A health functional food composition for preventing or improving hepatitis B, the health functional food composition comprising the nanovesicles of claim 1, the nanovesicles being loaded with a hepatitis B treatment drug as an active ingredient.
